(19) United States
(12) Patent Application Publication
(10) Pub. No.: US 2023/0200779 A1
(43) Pub. Date: Jun. 29, 2023

(54) ULTRASOUND SYSTEM AND CONTROL METHOD OF ULTRASOUND SYSTEM

(71) Applicant: FUJIFILM Corporation, Tokyo (JP)

(72) Inventor: Riko KOSHINO, Kanagawa (JP)

(73) Assignee: FUJIFILM Corporation, Tokyo (JP)

(21) Appl. No.: 18/177,564

(22) Filed: Mar. 2, 2023

Related U.S. Application Data
(63) Continuation of application No. PCT/JP2021/028918, filed on Aug. 4, 2021.

(30) Foreign Application Priority Data
Sep. 23, 2020 (JP) 2020-158399

(51) Int. Cl. A61B 8/00; A61B 8/08

(52) U.S. Cl. CPC A61B 8/4254; A61B 8/0825; A61B 8/4209; A61B 8/463

(57) ABSTRACT

In an ultrasound system and a control method of the ultrasound system, a first position of an ultrasound probe on a nipple of a subject and a second position of the ultrasound probe on epidermis above a region of interest in a three-dimensional space are acquired, and the region of interest and the epidermis in an ultrasound image are specified. A first linear distance L1 from the first position to the second position in the three-dimensional space and a second linear distance L2 from the region of interest to the epidermis above the region of interest in the ultrasound image are calculated. Then, a third linear distance L3 from the nipple to the region of interest in the ultrasound image is calculated on the basis of the first linear distance L1 and the second linear distance L2.

### CROSS-REFERENCE TO RELATED APPLICATIONS

This application is a Continuation of PCT International Application No. PCT/JP2021/028918 filed on Aug. 4, 2021, which claims priority under 35 U.S.C. § 119(a) to Japanese Patent Application No. 2020-158399 filed on Sep. 23, 2020. The above applications are hereby expressly incorporated by reference, in their entirety, into the present application.

### BACKGROUND OF THE INVENTION

### 1. Field of the Invention

The present invention relates to an ultrasound system and a control method of the ultrasound system which have a function of calculating a linear distance from a nipple in an ultrasound image of a breast to a region of interest of the breast with a possibility of being a lesion part.

### 2. Description of the Related Art

Ultrasonography for screening and diagnosis of mammary glands is often performed after mammography examination. For example, in a case where a region of interest of a breast is found by the mammography examination, by ultrasonography, a region of interest of a breast in an ultrasound image, which corresponds to the region of interest of the breast in a mammography image is specified, and a discrimination is performed as to whether the region of interest of the breast is a lesion part or not, for example, is a cyst or not, or is malignant lymphoma or not. In the ultrasonography, there are a case where the discrimination is performed by specifying only the region of interest of the breast in the ultrasound image, which corresponds to the region of interest of the breast in the mammography image, and a case where the discrimination is performed by specifying all the regions of interest of the breast in the ultrasound image by examining the entire breast.

Here, there are JP2017-86896A and the like as the documents in the related art as references for the present invention.

JP2017-86896A discloses that in a medical examination using both the mammography image and the ultrasound image, information on a position of a region of interest in a breast, a size of the region of interest, a size of the breast, and the like is stored on the basis of the mammography image obtained by imaging the breast of a subject, and a setting condition in an ultrasound image diagnosis is obtained on the basis of the information on the position of the region of interest, the size of the region of interest, the size of the breast, and the like obtained in the mammography image.

### SUMMARY OF THE INVENTION

It is useful to specify a region of interest of a breast in an ultrasound image using information on a feature site of the breast in the mammography image such as the nipple, epidermis, pectoralis major muscle, or chest wall, a distance from a compression plate and a radiation detector to the region of interest of the breast at the time of the mammography examination, and a size (long diameter and short diameter) of the region of interest of the breast.

In a case where a diagnostic report of the ultrasonography is created, it is necessary to describe the positional information or the like of the region of interest of the breast according to the guidelines of Breast Imaging Reporting and Data System (BI-RADS). In this case, as the positional information of the region of interest of the breast, information such as a position mark representing a position of an ultrasound probe on a schema diagram of the breast, a clock position representing the position of the ultrasound probe, for example, the 3 o'clock direction, and a linear distance from the nipple to the region of interest of the breast is often used.

However, JP2017-86896A does not disclose how to obtain the linear distance from the nipple to the region of interest of the breast, which is important as the positional information of the region of interest of the breast in the ultrasound image.

For example, even in a case where a region of interest of the breast is present at a position away from the nipple and the nipple is not displayed in the normal ultrasound image including the region of interest of the breast, it is possible to calculate the linear distance from the nipple to the epidermis above the region of interest of the breast by detecting the position of the ultrasound probe on the nipple and the position of the ultrasound probe on the epidermis above the region of interest of the breast using, for example, a magnetic sensor.

However, since the magnetic sensor detects the position of the ultrasound probe on the epidermis, the linear distance from the nipple to the epidermis above the region of interest of the breast can be calculated on the basis of the position of the ultrasound probe, but the linear distance from the nipple to the region of interest of the breast cannot be acquired. Therefore, there is a problem that an error between the linear distance from the nipple to the region of interest of the breast and the distance from the nipple to the epidermis above the region of interest of the breast is increased as the region of interest of the breast approaches the pectoralis major muscle or chest wall side rather than the epidermis side.

An object of the present invention is to provide an ultrasound system and a control method of the ultrasound system which can accurately calculate a linear distance from a nipple to a region of interest of a breast in an ultrasound image.

In order to achieve the object, an aspect of the present invention provides an ultrasound system comprising an ultrasound probe; a position sensor that outputs a position detection signal for detecting a position of the ultrasound probe in a three-dimensional space; an image generation unit that generates an ultrasound image including epidermis and a region of interest of a breast of a subject, from a reception signal obtained by performing transmission and reception of an ultrasound beam with respect to the region of interest using the ultrasound probe, on the epidermis above the region of interest; a position acquisition unit that acquires a first position of the ultrasound probe on a nipple of the subject and a second position of the ultrasound probe on the epidermis above the region of interest in the three-dimensional space, which are detected on the basis of the position detection signal; a site specifying unit that specifies the epidermis and the region of interest in the ultrasound image; a first distance calculation unit that calculates a first linear distance L1 from the first position to the second position in the three-dimensional space; a second distance calculation unit that calculates a second linear distance L2 from the region of interest to the epidermis above the region of interest in the ultrasound image; and a third distance calculation unit that calculates a third linear distance L3 from the nipple to the region of interest in the ultrasound image on the basis of the first linear distance L1 and the second linear distance L2.

Here, it is preferable that, in a case where, by a first straight line from the first position to the second position, a second straight line from the region of interest to the epidermis above the region of interest in the ultrasound image, and a third straight line from the nipple to the region of interest in the ultrasound image in the three-dimensional space, a right triangle in which an angle formed by the first straight line and the second straight line is substantially a right angle is formed, the third distance calculation unit uses the Pythagoras' theorem to calculate the third linear distance L3 on the basis of the first linear distance L1 and the second linear distance L2.

It is preferable that, in a case where, by a first straight line from the first position to the second position, a second straight line from the region of interest to the epidermis above the region of interest in the ultrasound image, and a third straight line from the nipple to the region of interest in the ultrasound image in the three-dimensional space, a right triangle in which an angle formed by the second straight line and the third straight line is substantially a right angle is formed, the third distance calculation unit uses the Pythagoras' theorem to calculate the third linear distance L3 on the basis of the first linear distance L1 and the second linear distance L2.

It is preferable that the position sensor outputs an angle detection signal for detecting an angle of the ultrasound probe on the epidermis above the region of interest with respect to a vertical direction in the three-dimensional space, the position acquisition unit acquires a first angle θ1 of the ultrasound probe on the nipple with respect to the vertical direction and a second angle θ2 of the ultrasound probe on the epidermis above the region of interest with respect to the vertical direction in the three-dimensional space, which are detected on the basis of the angle detection signal, and in a case where, by a first straight line from the first position to the second position, a fourth straight line extending from the nipple toward an inside of the subject at the first angle θ1, and a fifth straight line extending from the epidermis above the region of interest toward the inside of the subject at the second angle θ2, an isosceles triangle in which distances of the fourth straight line and the fifth straight line are equal is formed, and by the third straight line, a sixth straight line extending perpendicularly from the nipple to the fifth straight line, and a seventh straight line from an intersection between the fifth straight line and the sixth straight line to the region of interest, a right triangle in which an angle formed by the sixth straight line and the seventh straight line is a right angle is formed, the third distance calculation unit uses the Pythagoras' theorem to calculate the third linear distance L3 on the basis of the first linear distance L1, the second linear distance L2, the first angle θ1, and the second angle θ2.

It is preferable that the position sensor is a magnetic sensor, a GPS sensor, or an optical sensor.

It is preferable that the ultrasound system further includes a monitor; and a display control unit that displays information on the third linear distance L3 on the monitor by superimposing the information on the ultrasound image including the region of interest.

It is preferable that the display control unit further displays information on the second linear distance L2 on the monitor by superimposing the information on the ultrasound image including the region of interest.

It is preferable that the site specifying unit specifies a pectoralis major muscle or chest wall of the subject in the ultrasound image, the ultrasound system further includes a fourth distance calculation unit that calculates a fourth linear distance L4 from the region of interest to the pectoralis major muscle or chest wall in the ultrasound image, and the display control unit further displays information on the fourth linear distance L4 on the monitor by superimposing the information on the ultrasound image including the region of interest.

It is preferable that the ultrasound system further includes an ultrasound diagnostic apparatus; and a server, in which the ultrasound diagnostic apparatus includes the ultrasound probe, the position sensor, and the image generation unit, and the server includes the third distance calculation unit.

It is preferable that the ultrasound system further includes an input device that receives an instruction input from a user, in which the site specifying unit specifies at least one of the region of interest or the epidermis in the ultrasound image on the basis of the instruction input from the user.

It is preferable that the ultrasound system further includes an image analysis unit that analyzes the ultrasound image, in which the site specifying unit specifies at least one of the region of interest or the epidermis in the ultrasound image on the basis of an analysis result of the ultrasound image.

It is preferable that the site specifying unit has a determination model that has learned, using learning ultrasound images including a region of interest of a breast of a subject as teacher data, a relationship between the learning ultrasound image and the region of interest and epidermis included in the learning ultrasound image, and the determination model uses the ultrasound image as an input, and specifies at least one of the region of interest or the epidermis in the ultrasound image.

It is preferable that information on the third linear distance L3 is transmitted to a picture archiving and communication system, and is displayed on a display device of the picture archiving and communication system.

Further, another aspect of the present invention provides a control method of an ultrasound system, the control method including: outputting a position detection signal for detecting a position of an ultrasound probe in a three-dimensional space; generating an ultrasound image including a region of interest and epidermis, from a reception signal obtained by performing transmission and reception of an ultrasound beam with respect to the region of interest using the ultrasound probe, on the epidermis above the region of interest of a breast of a subject; acquiring a first position of the ultrasound probe on a nipple of the subject and a second position of the ultrasound probe on the epidermis above the region of interest in the three-dimensional space, which are detected on the basis of the position detection signal; specifying the epidermis and the region of interest in the ultrasound image; calculating a first linear distance L1 from the first position to the second position in the three-dimensional space; calculating a second linear distance L2 from the region of interest to the epidermis above the region of interest in the ultrasound image; and calculating a third linear distance L3 from the nipple to the region of interest in the ultrasound image on the basis of the first linear distance L1 and the second linear distance L2.

According to the present invention, with the configuration described above, the third linear distance L3 from the nipple to the region of interest of the breast in the ultrasound image can be accurately calculated on the basis of the first linear distance L1 from the first position of the ultrasound probe 1 on the nipple to the second position of the ultrasound probe 1 on the epidermis above the region of interest of the breast in the three-dimensional space, and the second linear distance L2 from the region of interest of the breast to the epidermis above the region of interest of the breast in the ultrasound image.

### BRIEF DESCRIPTION OF THE DRAWINGS

FIG. 1 is a block diagram of an embodiment illustrating a configuration of an ultrasound system of the present invention.

FIG. 2 is a block diagram of an embodiment illustrating a configuration of an ultrasound diagnostic apparatus.

FIG. 3 is a block diagram of an embodiment illustrating a configuration of a transmission and reception circuit.

FIG. 4 is a block diagram of an embodiment illustrating a configuration of an image generation unit.

FIG. 5 is a block diagram of an embodiment illustrating a configuration of a distance calculation unit.

FIG. 6 is a block diagram of an embodiment illustrating a configuration of a position detection device.

FIG. 7 is a flowchart of an embodiment illustrating an operation of an ultrasound system in a case of capturing an ultrasound image.

FIG. 8 is a flowchart of an embodiment illustrating an operation of an ultrasound system in a case of calculating a linear distance from a nipple to a region of interest of a breast in an ultrasound image.

FIG. 9 is a conceptual diagram of an embodiment illustrating a display screen of a monitor.

FIG. 10 is a block diagram of an embodiment illustrating a configuration of a server.

FIG. 11 is a conceptual diagram of an example illustrating a relationship among a first linear distance L1, a second linear distance L2, and a third linear distance L3.

FIG. 12 is a conceptual diagram of another example illustrating a relationship among a first linear distance L1, a second linear distance L2, and a third linear distance L3.

FIG. 13 is a conceptual diagram of another example illustrating a relationship among a first linear distance L1, a second linear distance L2, and a third linear distance L3.

### DESCRIPTION OF THE PREFERRED EMBODIMENTS

Hereinafter, an ultrasound system and a control method of the ultrasound system according to the present invention will be described in detail on the basis of preferred embodiments illustrated in the accompanying drawings.

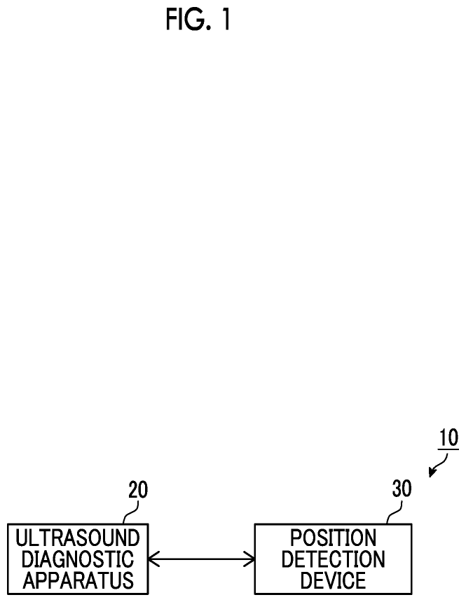

FIG. 1 is a block diagram of an embodiment illustrating a configuration of an ultrasound system of the present invention. An ultrasound system 10 illustrated in FIG. 1 includes an ultrasound diagnostic apparatus 20 and a position detection device 30. The ultrasound diagnostic apparatus 20 and the position detection device 30 are connected to each other, and thereby data can be bidirectionally delivered. The ultrasound diagnostic apparatus 20 and the position detection device 30 may be connected to each other via a network such as a local network in a hospital, for example.

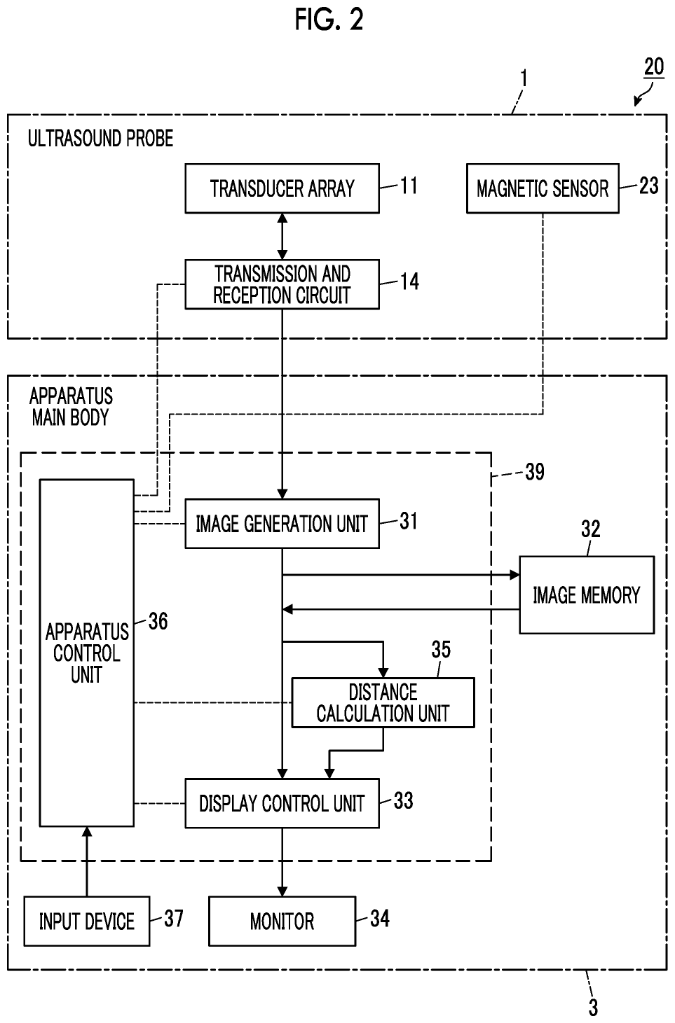

FIG. 2 is a block diagram of an embodiment illustrating a configuration of the ultrasound diagnostic apparatus 20. The ultrasound diagnostic apparatus 20 illustrated in FIG. 2 includes an ultrasound probe 1, and an apparatus main body 3 connected to the ultrasound probe 1.

The ultrasound probe 1 scans a subject using an ultrasound beam, and outputs a sound ray signal corresponding to an ultrasound image. As illustrated in FIG. 2, the ultrasound probe 1 includes a transducer array 11, a transmission and reception circuit 14, and a magnetic sensor 23. The transducer array 11 and the transmission and reception circuit 14 are bidirectionally connected to each other. Further, an apparatus control unit 36 to be described later is connected to the transmission and reception circuit 14 and the magnetic sensor 23.

The transducer array 11 has a plurality of ultrasonic transducers arranged in a one-dimensional or two-dimensional manner. According to a drive signal supplied from the transmission and reception circuit 14, each of the transducers transmits an ultrasonic wave and receives a reflected wave from the subject to output an analog reception signal.

For example, each transducer is formed by using an element in which electrodes are formed at both ends of a piezoelectric body consisting of piezoelectric ceramic represented by lead zirconate titanate (PZT), a polymer piezoelectric element represented by poly vinylidene di fluoride (PVDF), piezoelectric single crystal represented by lead magnesium niobate-lead titanate (PMN-PT), or the like.

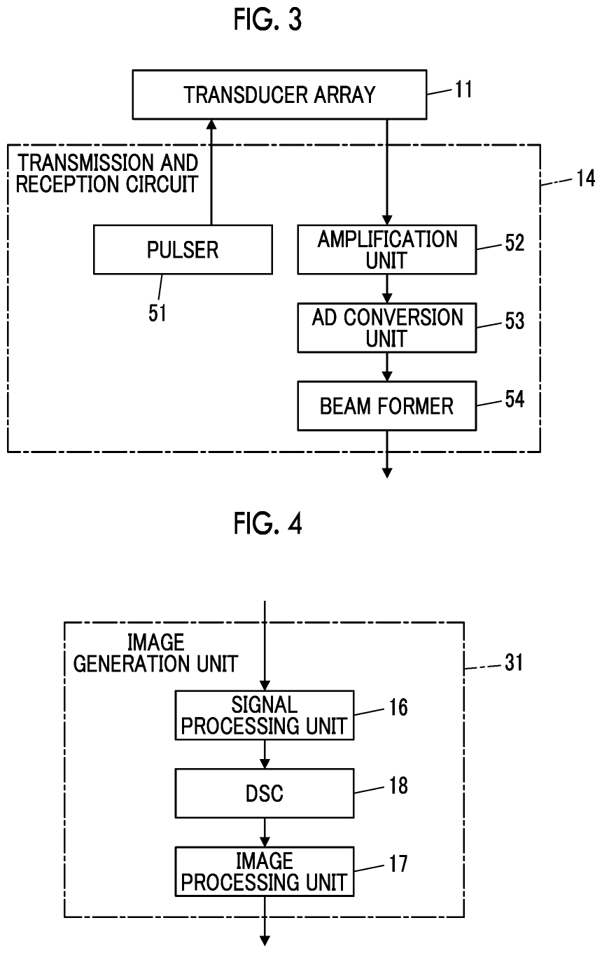

The transmission and reception circuit 14 causes the transducer array 11 to transmit the ultrasonic wave, and performs reception focusing processing on the reception signal output from the transducer array 11 that has received the ultrasound echo to generate a sound ray signal, under the control of the apparatus control unit 36. As illustrated in FIG. 3, the transmission and reception circuit 14 has a pulser 51 connected to the transducer array 11, and an amplification unit 52, an analog digital (AD) conversion unit 53, and a beam former 54 that are sequentially connected in series from the transducer array 11.

The pulser 51 includes, for example, a plurality of pulse generators, and the pulser 51 adjusts the amount of delay of each drive signal so that ultrasonic waves transmitted from the plurality of transducers of the transducer array 11 form an ultrasound beam on the basis of a transmission delay pattern selected by the apparatus control unit 36, and supplies the obtained signals to the plurality of transducers. Thus, in a case where a pulsed or continuous-wave voltage is applied to the electrodes of the transducers of the transducer array 11, the piezoelectric body expands and contracts to generate pulsed or continuous-wave ultrasonic waves from each transducer. From the combined wave of these ultrasonic waves, an ultrasound beam is formed.

The transmitted ultrasound beam is reflected by a target, for example, a site of the subject, and propagates toward the transducer array 11 of the ultrasound probe 1. Each transducer constituting the transducer array 11 expands and contracts by receiving the ultrasound echo propagating toward the transducer array 11 in this manner, to generate the reception signal that is an electric signal, and outputs the reception signal to the amplification unit 52.

The amplification unit 52 amplifies the signals input from each transducer constituting the transducer array 11, and transmits the amplified signals to the AD conversion unit 53. The AD conversion unit 53 converts the analog signal transmitted from the amplification unit 52 into digital reception data, and outputs the reception data to the beam former 54.

The beam former 54 performs so-called reception focusing processing in which addition is performed by giving delays to respective pieces of the reception data converted by the AD conversion unit 53 according to a sound speed distribution or a sound speed set on the basis of a reception delay pattern selected by the apparatus control unit 36. Through the reception focusing processing, a sound ray signal in which each piece of the reception data converted by the AD conversion unit 53 is phased and added and the focus of the ultrasound echo is narrowed is generated.

The magnetic sensor 23 is a position sensor that outputs a position detection signal for the position detection device 30 to detect a position (three-dimensional coordinate position) of the ultrasound probe 1 in a three-dimensional space of the magnetic field using magnetism, under the control of the apparatus control unit 36. The magnetic sensor 23 outputs an angle detection signal for the position detection device 30 to detect an angle of the ultrasound probe 1 with respect to a vertical direction in the three-dimensional space using magnetism.

Next, the apparatus main body 3 displays the ultrasound image on the basis of the sound ray signal generated by the ultrasound probe 1. As illustrated in FIG. 2, the apparatus main body 3 includes an image generation unit 31, an image memory 32, a distance calculation unit 35, a display control unit 33, the apparatus control unit 36, a monitor (display unit) 34, and an input device 37.

The display control unit 33 and the monitor 34 are sequentially connected in series to the image generation unit 31. Each of the image memory 32 and the distance calculation unit 35 is connected to the image generation unit 31, and the display control unit 33 is connected to the image memory 32 and the distance calculation unit 35. The apparatus control unit 36 is connected to the transmission and reception circuit 14, the image generation unit 31, the display control unit 33, and the distance calculation unit 35, and the input device 37 is connected to the apparatus control unit 36.

The image generation unit 31 generates the ultrasound image (ultrasound image signal) on the basis of the sound ray signal generated by the transmission and reception circuit 14 under the control of the apparatus control unit 36. As illustrated in FIG. 4, the image generation unit 31 has a configuration in which a signal processing unit 16, a digital scan converter (DSC) 18, and an image processing unit 17 are sequentially connected in series.

The signal processing unit 16 generates image information data corresponding to the ultrasound image on the basis of the sound ray signal generated by the transmission and reception circuit 14. More specifically, the signal processing unit 16 generates the image information data representing tomographic image information regarding tissues inside the subject, by performing envelope detection processing after signal processing, for example, correcting the attenuation of the sound ray signal generated by the beam former 54 of the transmission and reception circuit 14, which is caused by the propagation distance according to the depth of the reflection position of the ultrasonic wave.

The DSC 18 raster-converts the image information data generated by the signal processing unit 16 into an image signal according to a normal television signal scanning method.

The image processing unit 17 performs various kinds of image processing such as brightness correction, gradation correction, sharpness correction, image size correction, refresh rate correction, scanning frequency correction, and color correction according to a display format of the monitor 34, on the image signal input from the DSC 18 to generate the ultrasound image (ultrasound image signal), and then outputs the generated ultrasound image to the display control unit 33 and the image memory 32.

In the present embodiment, the image generation unit 31 generates an ultrasound image including a region of interest of the breast and the epidermis from the reception signal obtained by performing transmission and reception of the ultrasound beams with respect to the region of interest of the breast of the subject using the ultrasound probe 1 (more precisely, transducer array 11) on the epidermis above the region of interest of the breast of the subject, in other words, from the sound ray signal generated from the reception signal by the transmission and reception circuit 14.

The image memory 32 is a memory that stores ultrasound images (ultrasound image signal) of the series of a plurality of frames, which are generated for each diagnosis by the image generation unit 31. Here, as the image memory 32, recording media such as a flash memory, a hard disc drive (HDD), a solid state drive (SSD), a flexible disc (FD), a magneto-optical disc (MO disc), a magnetic tape (MT), a random access memory (RAM), a compact disc (CD), a digital versatile disc (DVD), a secure digital card (SD card), and a universal serial bus memory (USB memory), a server, or the like can be used.

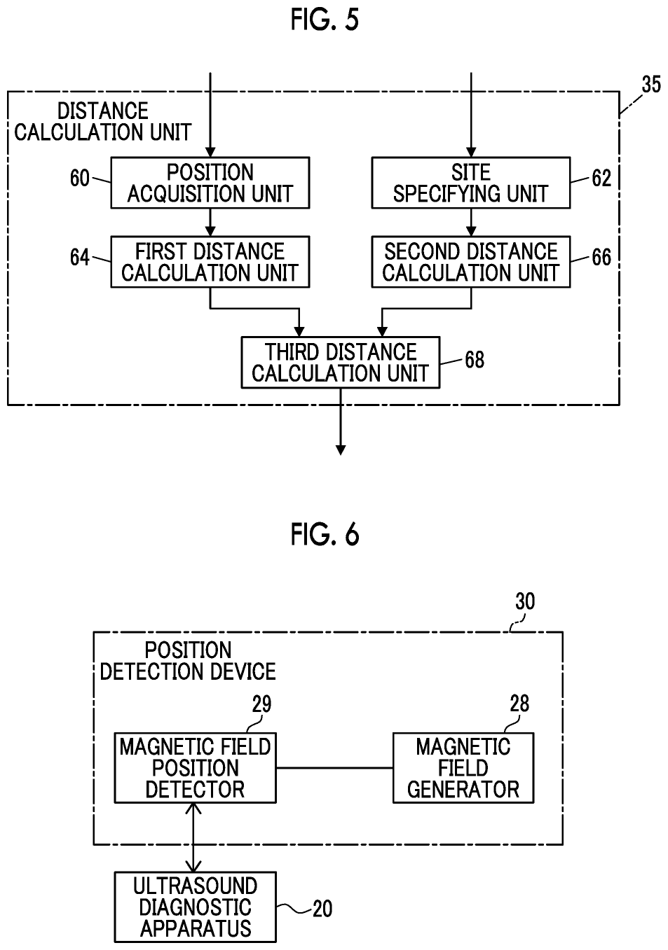

The distance calculation unit 35 calculates a linear distance or the like from the nipple to the region of interest of the breast under the control of the apparatus control unit 36. As illustrated in FIG. 5, the distance calculation unit 35 includes a position acquisition unit 60, a site specifying unit 62, a first distance calculation unit 64, a second distance calculation unit 66, and a third distance calculation unit 68.

The first distance calculation unit 64 is connected to the position acquisition unit 60, and the second distance calculation unit 66 is connected to the site specifying unit 62. Further, the first distance calculation unit 64 and the second distance calculation unit 66 are connected to the third distance calculation unit 68.

The position acquisition unit 60 acquires, from the position detection device 30, a first position of the ultrasound probe 1 on the nipple of the subject in the three-dimensional space, a second position of the ultrasound probe 1 on the epidermis above the region of interest of the breast in the three-dimensional space in a case of generating the ultrasound image (static image) including the region of interest of the breast, a first angle θ1 (absolute angle) of the ultrasound probe 1 with respect to the vertical direction on the nipple in the three-dimensional space, a second angle θ2 (absolute angle) of the ultrasound probe 1 with respect to the vertical direction on the epidermis above the region of interest of the breast in the three-dimensional space, and the like.

The site specifying unit 62 specifies the region of interest of the breast, the epidermis above the region of interest of the breast, the pectoralis major muscle, or the chest wall in the ultrasound image.

The site specifying unit 62 can specify at least one site in the ultrasound image on the basis of an instruction input from the user. Further, an image analysis unit that analyzes the ultrasound image may be provided, and the site specifying unit 62 may specify at least one site in the ultrasound image on the basis of an analysis result of the ultrasound image by the image analysis unit. A determination model may be provided, and the site specifying unit 62 may specify at least on site in the ultrasound image using the determination model.

here, the determination model is a trained model that has learned, using learning ultrasound images including the site of any subject such as the region of interest of the breast, the epidermis, the pectoralis major muscle, or the chest wall as teacher data, a relationship between the learning ultrasound image and each site included in the learning ultrasound image, for a plurality of pieces of the teacher data. The determination model uses an ultrasound image that is a determination target as an input, and outputs a determination result (prediction result) of each site included in the ultrasound image on the basis of the training result. That is, the site in the ultrasound image is specified by the determination model.

The first distance calculation unit 64 calculates the first linear distance L1 from the first position of the ultrasound probe 1 on the nipple to the second position of the ultrasound probe 1 on the epidermis above the region of interest of the breast in the three-dimensional space on the basis of the position acquired by the position acquisition unit 60.

The second distance calculation unit 66 calculates the second linear distance L2 from the region of interest of the breast to the epidermis above the region of interest of the breast in the ultrasound image on the basis of the site specified by the site specifying unit 62. Here, the position of the epidermis above the region of interest of the breast in the ultrasound image corresponds to the second position of the ultrasound probe 1 on the epidermis above the region of interest of the breast in the three-dimensional space in a case of generating the ultrasound image.

The third distance calculation unit 68 calculates the third linear distance L3 from the nipple to the region of interest of the breast in the ultrasound image on the basis of the first angle θ1 and the second angle θ2 acquired by the position acquisition unit 60 in addition to the first linear distance L1 calculated by the first distance calculation unit 64 and the second linear distance L2 calculated by the second distance calculation unit 66.

As illustrated in FIG. 11, in a case where the breast of the subject in a supine state is substantially flat, it is assumed that the ultrasound probe 1 is in contact with the epidermis of the breast in a direction substantially perpendicular to the epidermis, and, in the three-dimensional space, an angle of the ultrasound probe 1 on the nipple with respect to the epidermis of the breast and an angle of the ultrasound probe 1 on the epidermis above the region of interest of the breast with respect to the epidermis of the breast are substantially right angles. In this case, as illustrated in FIG. 11, by a first straight line from the first position to the second position in the three-dimensional space, a second straight line from the region of interest of the breast to the epidermis above the region of interest in the ultrasound image, and a third straight line from the nipple to the region of interest of the breast in the ultrasound image, a right triangle in which an angle formed by the first straight line and the second straight line is a substantially right angle is formed. Accordingly, the third distance calculation unit 68 uses the Pythagoras' theorem to calculate the third linear distance L3 by following Expression (1) on the basis of the first linear distance L1 and the second linear distance L2.

L3=√(L2²+L2²) Expression (1)

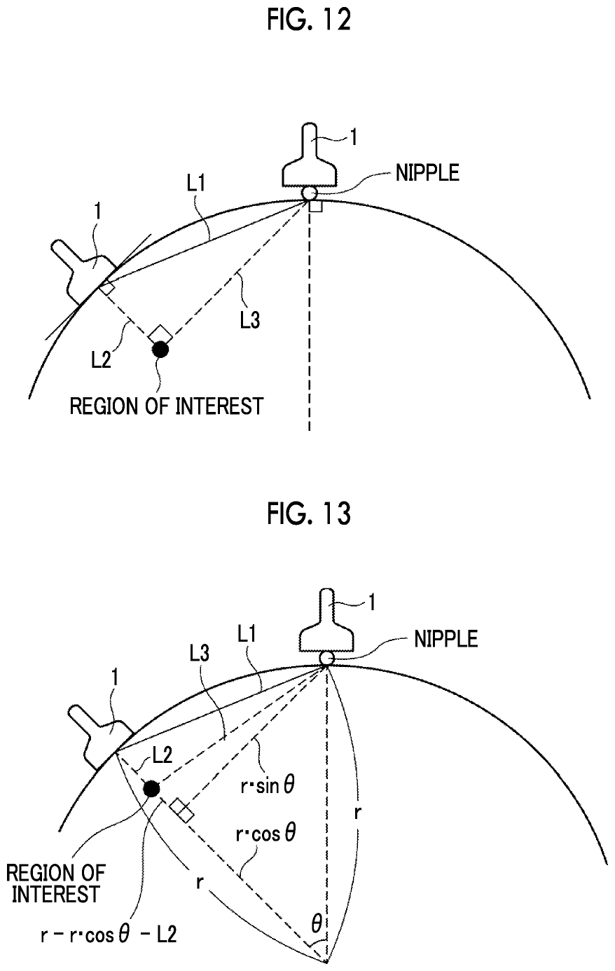

Further, as illustrated in FIG. 12, in a case where the breast of the subject in a supine state is convex, it is similarly assumed that the ultrasound probe 1 is in contact with the epidermis of the breast in a direction substantially perpendicular to the epidermis, and, in the three-dimensional space, an angle of the ultrasound probe 1 on the nipple with respect to the epidermis of the breast and an angle of the ultrasound probe 1 on the epidermis above the region of interest of the breast with respect to the epidermis of the breast are substantially right angles. In this case, as illustrated in FIG. 12, assuming that a right triangle in which an angle between the second straight line and the third straight line is a substantially right angle is formed by the first straight line, the second straight line, and the third straight line, the third distance calculation unit 68 uses the Pythagoras' theorem to calculate the third linear distance L3 by following Expression (2) on the basis of the first linear distance L1 and the second linear distance L2.

L3=√(L1²−L2²) Expression (2)

Further, as illustrated in FIG. 13, in a case where the breast of the subject in a supine state is convex, it is assumed that the ultrasound probe 1 is in contact with the epidermis of the breast by being inclined from a direction substantially perpendicular to the epidermis, and, in the three-dimensional space, an angle of the ultrasound probe 1 on the nipple with respect to the vertical direction and an angle of the ultrasound probe 1 on the epidermis above the region of interest of the breast with respect to the vertical direction are the first angle θ1 and the second angle θ2, respectively. In this case, as illustrated in FIG. 13, by the first straight line, a fourth straight line extending from the nipple toward the inside of the subject at the first angle θ1, and a fifth straight line extending from the epidermis above the region of interest of the breast toward the inside of the subject at the second angle θ2, an isosceles triangle in which distances of the fourth straight line and the fifth straight line are equal is formed. Further, by the third straight line, a sixth straight line extending perpendicularly from the nipple to the fifth straight line, and a seventh straight line from an intersection between the fifth straight line and the sixth straight line to the region of interest of the breast, a right triangle in which an angle formed by the sixth straight line and the seventh straight line is a right angle is formed. Accordingly, the third distance calculation unit 68 uses the Pythagoras' theorem to calculate the third linear distance L3 by following Expression (3) on the basis of the first linear distance L1, the second linear distance L2, the first angle θ1, and the second angle θ2.

L3=√{(r·sinθ)²+(r−r·cosθ−L2)²} Expression (3)

Here, r·sinθ is a sixth linear distance L6, and r−r·cosθ−L2 is a seventh linear distance L7. θ is the difference angle between the first angle θ1 and the second angle θ2. r is a fourth linear distance from the nipple to the intersection between the fourth straight line and the fifth straight line, and a fifth linear distance from the epidermis above the region of interest of the breast to the intersection between the fourth straight line and the fifth straight line, and Expression (4) is established on the basis of the first linear distance L1 and the difference angle θ.

L2=2r·sin(θ/2) Expression (4)

The display control unit 33 displays various kinds of information on the monitor 34 under the control of the apparatus control unit 36. For example, the display control unit 33 performs predetermined processing on the ultrasound image held in the image memory 32, and displays the processed ultrasound image on the monitor 34. The display control unit 33 displays information on the third linear distance L3 and the like on the monitor 34 by superimposing the information on the ultrasound image including the region of interest of the breast.

The apparatus control unit 36 controls each unit of the apparatus main body 3 on the basis of a program stored in advance and an instruction or the like of the user input from the input device 37. More specifically, the apparatus control unit 36 controls the display control unit 33 such that the ultrasound image is displayed on the monitor 34. The apparatus control unit 36 controls the distance calculation unit 35 such that the third linear distance L3 from the nipple to the region of interest of the breast and the like are calculated.

The image generation unit 31, the display control unit 33, the distance calculation unit 35, and the apparatus control unit 36 constitute a terminal-side processor 39.

The monitor 34 displays various kinds of information under the control of the display control unit 33. The monitor 34 displays the ultrasound image, the information on the third linear distance L3 from the nipple to the region of interest of the breast, and the like. Examples of the monitor 34 include a display device such as a liquid crystal display (LCD), and an organic electroluminescence (EL) display.

The input device 37 receives various instructions input from the user, and includes various buttons, and a touch panel or the like through which various instructions are input by the user performing a touch operation.

Next, the position detection device 30 detects the position of the ultrasound probe 1 on the epidermis of the breast in the three-dimensional space, more precisely, the position of the magnetic sensor 23, and includes a magnetic field generator 28, and a magnetic field position detector 29, as illustrated in FIG. 6. The magnetic field generator 28 is connected to the magnetic field position detector 29, and the magnetic field position detector 29 and the ultrasound diagnostic apparatus 20 are connected to each other.

During the ultrasonography, the position detection device 30 detects the position of the ultrasound probe 1 on the epidermis of the breast, that is, the position of the magnetic sensor 23, and the angle of the ultrasound probe 1 with respect to the vertical direction (inclination of the ultrasound probe 1 with respect to the vertical direction) on the basis of the position detection signal and the angle detection signal output from the magnetic sensor 23 of the ultrasound probe 1, by the magnetic field position detector 29, in the three-dimensional space of the magnetic field generated by the magnetic field generator 28.

The position of the ultrasound probe 1 can be detected by using not only the magnetic sensor, but also a Global Positioning System (GPS) sensor, an optical sensor, or the like as the position sensor. In a case where the GPS sensor is used as the position sensor, a position detection device that detects the position of the ultrasound probe 1 using the GPS, and detects the angle of the ultrasound probe 1 using the gyro sensor is used. On the other hand, in a case where the optical sensor is used as the position sensor, a position detection device that detects the position and angle of the ultrasound probe 1 using light is used.

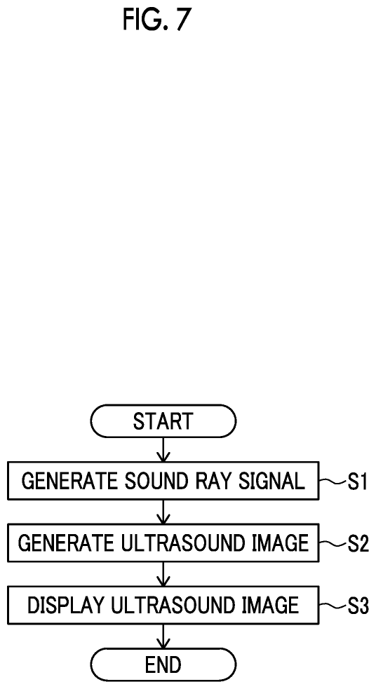

Next, the operation of the ultrasound system 10 in a case where the ultrasound image is captured will be described with reference to the flowchart of FIG. 7.

First, in a state where the ultrasound probe 1 is in contact with the epidermis of the breast of the subject, under the control of the apparatus control unit 36, the transmission of the ultrasonic waves is started by the transmission and reception circuit 14, and the sound ray signal is generated (Step S1).

That is, the ultrasound beams are transmitted to the breast from a plurality of transducers of the transducer array 11 according to the drive signals from the pulser 51.

Ultrasound echoes from the breast based on the ultrasound beams transmitted from the pulser 51 are received by each transducer of the transducer array 11, and the reception signal as an analog signal is output from each transducer of the transducer array 11, which has received the ultrasound echo.

The reception signal as the analog signal output from each transducer of the transducer array 11 is amplified by the amplification unit 52, and is subjected to AD conversion by the AD conversion unit 53, and thereby the reception data is acquired.

By performing the reception focusing processing on the reception data by the beam former 54, the sound ray signal is generated.

Next, under the control of the apparatus control unit 36, the ultrasound image (ultrasound image signal) of the breast is generated by the image generation unit 31 on the basis of the sound ray signal generated by the beam former 54 of the transmission and reception circuit 14 (Step S2).

That is, the sound ray signal generated by the beam former 54 is subjected to various kinds of signal processing by the signal processing unit 16, and the image information data representing tomographic image information regarding tissues inside the subject is generated.

The image information data generated by the signal processing unit 16 is raster-converted by the DSC 18, and is further subjected to various kinds of image processing by the image processing unit 17, and thus the ultrasound image (ultrasound image signal) is generated.

The ultrasound image generated by the image processing unit 17 is held in the image memory 32.

Next, under the control of the apparatus control unit 36, predetermined processing is performed on the ultrasound image held in the image memory 32 by the display control unit 33, and the processed ultrasound image is displayed on the monitor 34 (Step S3).

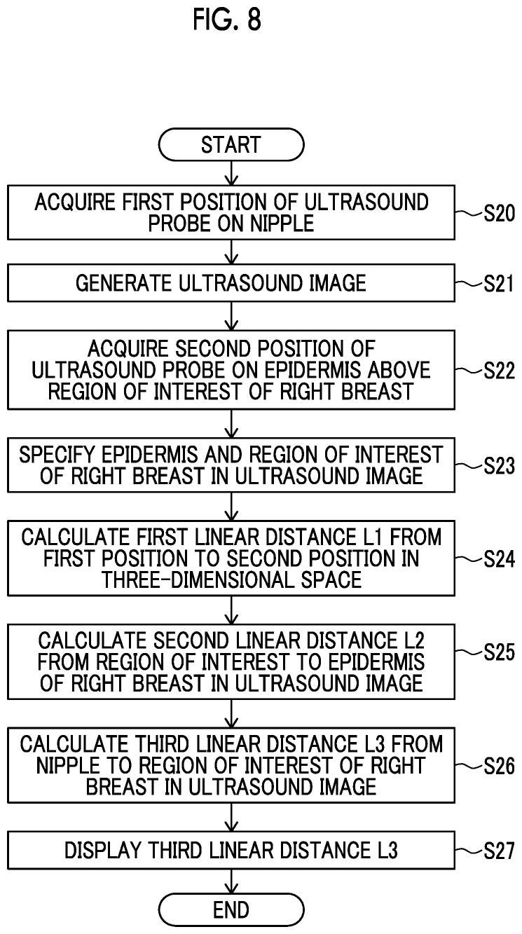

Next, the operation of the ultrasound system 10 in a case of calculating the linear distance from the nipple to the region of interest of the breast in the ultrasound image will be described with reference to the flowchart of FIG. 8. In the following description, it is assumed that a mammography examination is performed prior to the ultrasonography.

First, in a mammography apparatus, the breast of the subject, for example, in an upright state is pressed by a compression plate, and the breast pressed by the compression plate is irradiated with X-rays from an X-ray source. Then, the X-rays that have passed through the breast are detected by an X-ray detector, and a mammography image of the breast is generated from the detection signal detected by the X-ray detector. For example, mammography images (R_MLO image and R_CC image) in a mediolateral-oblique (MLO) direction and a cranio-caudal (CC) direction of the right breast are generated. Similarly, mammography images (L_MLO image and L_CC image) in the MLO direction and the CC direction of the left breast are generated.

In the ultrasound diagnostic apparatus 20, the mammography image is acquired from the mammography apparatus by the apparatus control unit 36 in response to the instruction from the user.

The ultrasound diagnostic apparatus 20 may acquire the mammography image from the server via the network instead of directly acquiring the mammography image from the mammography apparatus. The server is a picture archiving and communication system (PACS), or a computer or a workstation that manages the PACS, and stores and manages medical images such as a mammography image and an ultrasound image. In response to a request from the ultrasound diagnostic apparatus 20 or the like, the server provides the requested medical image from among the stored medical images, to the ultrasound diagnostic apparatus 20 or the like.

The ultrasound image (video) of the right breast of the subject in a supine state is generated by the image generation unit, and the mammography image and the ultrasound image of the right breast are displayed, for example, side by side on the monitor 34 by the display control unit 33.

In a state where the ultrasound probe 1 is in contact with the nipple substantially perpendicularly, a position detection button for detecting the position of the ultrasound probe 1 is pressed by the user. Accordingly, the first position of the ultrasound probe 1 on the nipple in the three-dimensional space of the magnetic field is detected by the position detection device 30 on the basis of the position detection signal output from the magnetic sensor 23 of the ultrasound probe 1. Then, the first position of the ultrasound probe 1 on the nipple is acquired from the position detection device 30 by the position acquisition unit 60 (Step S20).

In a state where the ultrasound probe 1 is in contact with the epidermis of the right breast substantially perpendicularly, the ultrasound probe 1 is moved from the nipple to the position of the epidermis above the region of interest of the right breast by the user.

In this case, the user watches the mammography image and the ultrasound image displayed on the monitor 34, and predicts the position of the region of interest of the right breast in the ultrasound image, which corresponds to the region of interest of the right breast in the mammography image. Then, the region of interest of the right breast can be specified by moving the ultrasound probe 1 to the position of the epidermis above the region of interest of the right breast in the ultrasound image, which corresponds to the region of interest of the right breast in the mammography image.

Alternatively, in a case of examining the entire right breast, the user repeats moving the ultrasound probe from the left side to the right side of the right breast a plurality of times while shifting the position in the up and down direction in order to prevent overlooking the region of interest of the right breast. The region of interest of the right breast can be specified by repeating moving the ultrasound probe from the upper side to the lower side of the right breast a plurality of times while shifting the position in the left and right direction.

In a state where the ultrasound probe 1 is in contact with the epidermis of the right breast substantially perpendicularly, the freeze button is pressed at the position of the epidermis above the region of interest of the right breast by the user. Accordingly, a first ultrasound image (static image) is generated by the image generation unit (Step S21). Further, for example, the orientation of the ultrasound probe 1 is rotated by 90 degrees at the position of the epidermis above the region of interest of the right breast, that is, the ultrasound probe 1 is directed to have an orientation orthogonal to the orientation of the ultrasound probe 1 at the time of the generation of the first ultrasound image, and the freeze button is pressed. Accordingly, a second ultrasound image (static image) is generated by the image generation unit (Step S21).

Further, in a case where the freeze button is pressed, accordingly, the second position of the ultrasound probe 1 on the epidermis above the region of interest of the right breast in the three-dimensional space is detected by the position detection device 30, and the second position of the ultrasound probe 1 on the epidermis above the region of interest of the right breast is acquired from the position detection device 30 by the position acquisition unit 60 (Step S22).

The second position of the ultrasound probe 1 on the epidermis above the region of interest of the right breast may be acquired in response to pressing the position detection button for detecting the position of the ultrasound probe 1 instead of the freeze button or in response to the stopping of the ultrasound probe 1 on the epidermis above the region of interest of the right breast for a few seconds.

The epidermis and the region of interest of the right breast in the ultrasound image are specified by the site specifying unit 62 (Step S23). The site specifying unit 62 may specify the epidermis and the region of interest of the right breast using any ultrasound image of two ultrasound images.

Subsequently, the first linear distance L1 from the first position of the ultrasound probe 1 on the nipple to the second position of the ultrasound probe 1 on the epidermis above the region of interest of the breast in the three-dimensional space is calculated by the first distance calculation unit 64 (Step S24).

The second linear distance L2 from the region of interest of the right breast to the epidermis above the region of interest of the right breast in the ultrasound image is calculated by the second distance calculation unit 66 (Step S25).

The third linear distance L3 from the nipple to the region of interest of the right breast in the ultrasound image is calculated by the third distance calculation unit 68 on the basis of the first linear distance L1 and the second linear distance L2 (Step S26).

Then, the information on the third linear distance L3 is displayed on the monitor 34 by being superimposed on the ultrasound image including the region of interest, by the display control unit 33 (Step S27). The ultrasound image which includes the region of interest and on which the information on the third linear distance L3 is superimposed is captured, and transmitted from the ultrasound diagnostic apparatus 20 to the PACS. The user can watch the information on the third linear distance L3 superimposed and displayed on the ultrasound image, on the display device (viewer) of the PACS, and enter the information of the third linear distance L3 in a diagnostic report of the ultrasonography.

Only the information on the third linear distance L3 may be transmitted from the ultrasound diagnostic apparatus 20 to the PACS, and the information on the third linear distance L3 may be superimposed on the ultrasound image including the region of interest, and displayed on the display device of the PACS.

In this manner, in the ultrasound system 10, the third linear distance L3 from the nipple to the region of interest of the breast in the ultrasound image can be accurately calculated on the basis of the first linear distance L1 from the first position of the ultrasound probe 1 on the nipple to the second position of the ultrasound probe 1 on the epidermis above the region of interest of the breast in the three-dimensional space, and the second linear distance L2 from the region of interest of the breast to the epidermis above the region of interest of the breast in the ultrasound image.

Not only the information on the third linear distance L3, but also information on the second linear distance L2 and further information on a fourth linear distance L4 from the region of interest of the breast to the pectoralis major muscle or chest wall may be displayed on the monitor 34.

In a case where the information on the fourth linear distance L4 is displayed on the monitor 34, the fourth distance calculation unit is provided, the pectoralis major muscle or chest wall in the ultrasound image is specified by the site specifying unit 62, the fourth linear distance L4 from the region of interest to the pectoralis major muscle or chest wall in the ultrasound image is calculated by the fourth distance calculation unit. Then, the information on the fourth linear distance L4 is displayed on the monitor 34 by being superimposed on the ultrasound image including the region of interest, by the display control unit 33.

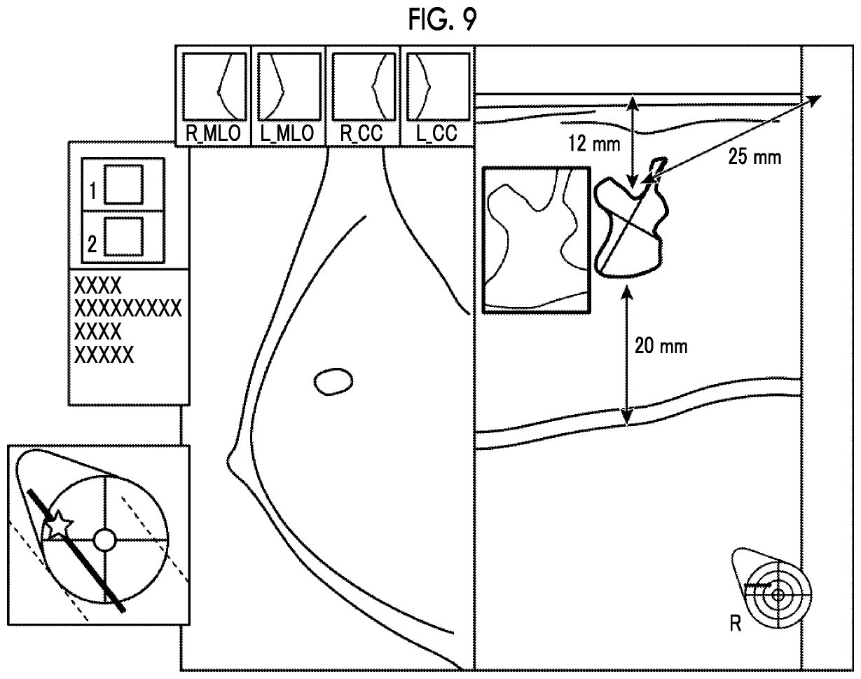

FIG. 9 is a conceptual diagram of an embodiment illustrating a display screen of the monitor. In a left display region of the display screen of the monitor 34 illustrated in FIG. 9, the R_MLO image is displayed.

Thumbnail images of the R_MLO image, the L_MLO image, the R_CC image, and the L_CC image are displayed in order from the left side in the upper portion of the left display region. The user can select one of the four thumbnail images to display the mammography image corresponding to the selected one thumbnail image in the left display region.

Two icon images corresponding to two regions of interest of the right breast in the R_MLO image displayed in the display region on the left side are displayed in the upper left portion of the left display region. The user can select one of the two icon images to display the finding for the region of interest corresponding to the selected one icon image.

A schematic view of the right breast indicating the cross section position of the breast and the position of the region of interest of the breast corresponding to the R_MLO image displayed in the display region on the left side is displayed in the lower left portion of the left display region.

In a right display region of the display screen of the monitor 34 illustrated in FIG. 9, the ultrasound image including the epidermis and the region of interest of the right breast is displayed.

An enlarged view of the region of interest of the right breast in the ultrasound image of the right breast displayed in the right display region is displayed in a window region on the upper left portion of the right display region.

A schematic view of the right breast indicating the cross section position corresponding to the ultrasound image of the right breast displayed in the right display region and the orientation of the ultrasound probe 1 is displayed on the lower right portion of the right display region.

As illustrated in FIG. 9, in the ultrasound image of the right breast displayed in the right display region, in addition to the information on the third linear distance L3 from the nipple to the region of interest of the right breast in the ultrasound image, the information on the second linear distance L2 from the region of interest of the right breast to the epidermis above the region of interest of the right breast, and the information on the fourth linear distance L4 from the region of interest of the right breast to the chest wall are displayed on the monitor 34 by being superimposed on the ultrasound image including the region of interest of the right breast under the control of the display control unit 33.

As illustrated in FIG. 9, as the information on the third linear distance L3, a two sided arrow connecting from the nipple to the region of interest of the right breast in the ultrasound image, and 25 mm that is a distance from the nipple to the region of interest of the right breast are displayed as the annotation on the monitor 34. The information on the second linear distance L2 and the information on the fourth linear distance L4 are similarly displayed. The display as the annotation can be turned on or off in response to an instruction from the user, for example, so as not to interfere with the interpretation of the ultrasound image by the user.

As the third linear distance L3, the linear distance from the nipple to the intersection of the long diameter and the short diameter of the region of interest of the right breast in the ultrasound image can be used. Alternatively, the linear distance from the nipple to the center point or centroid of the region of interest of the right breast, the linear distance from the nipple to the frame surrounding the region of interest of the right breast, or the like may be used. The third linear distance L3 has been described, but the same applies to the second linear distance L2 and the fourth linear distance L4.

A server may be provided, and the third linear distance L3 from the nipple to the region of interest in the ultrasound image may be calculated in the server instead of the ultrasound diagnostic apparatus 20. In this case, the server may be a PACS server, for example. As another example, the server may be a separate computer or workstation connected to the network.

In this case, the ultrasound diagnostic apparatus 20 includes constituents other than the distance calculation unit 35 illustrated in FIG. 2, for example, the ultrasound probe 1, the magnetic sensor 23, and the image generation unit.

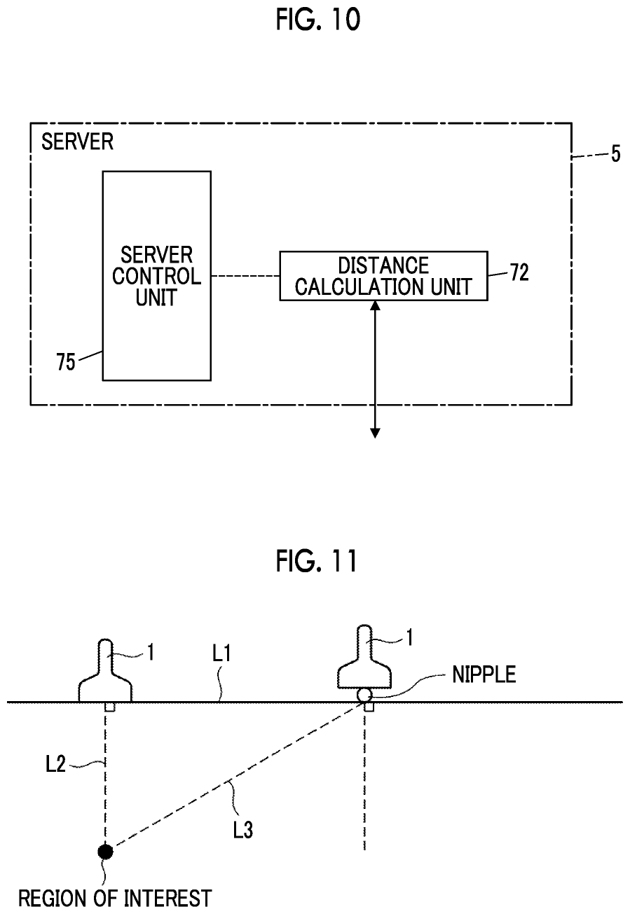

On the other hand, as illustrated in FIG. 10, a server 5 includes a distance calculation unit 72, and a server control unit 75.

The server 5 and the apparatus main body 3 are connected via the network, and thereby data can be bidirectionally delivered.

The distance calculation unit 72 is the same as the distance calculation unit 35 of the apparatus main body 3 of the ultrasound diagnostic apparatus 20, but is operated under the control of the server control unit 75.

The server control unit 75 controls each unit of the server 5 on the basis of a program and the like stored in advance. More specifically, the server control unit 75 controls the distance calculation unit 72 such that the third linear distance L3 from the nipple to the region of interest in the ultrasound image is calculated.

In the server 5, in a case of calculating the third linear distance L3, the ultrasound image is received from the ultrasound diagnostic apparatus 20 under the control of the server control unit 75. The operation of the distance calculation unit 72 is similar to the operation in a case where the distance calculation unit 35 calculates the third linear distance L3 in the ultrasound diagnostic apparatus 20. In the server 5, in a case where the third linear distance L3 is calculated, the information on the third linear distance L3 is transmitted from the server 5 to the ultrasound diagnostic apparatus 20.

Then, in the ultrasound diagnostic apparatus 20, the information on the third linear distance L3 is displayed on the monitor 34 by being superimposed on the ultrasound image including the region of interest, by the display control unit 33. Alternatively, the ultrasound image which includes the region of interest and on which the information on the third linear distance L3 is superimposed may be transmitted from the server 5 to the PACS separate from the server, and may be displayed on the display device (viewer) of the PACS. Further, only the information on the third linear distance L3 may be transmitted from the server 5 to the PACS, and the information on the third linear distance L3 may be superimposed on the ultrasound image including the region of interest, and displayed on the display device of the PACS.

In a case of calculating the third linear distance L3, the server 5 does not necessarily include the distance calculation unit 72, and the server 5 may include at least the third distance calculation unit of the distance calculation unit 72. That is, at least one of the position acquisition unit, the first distance calculation unit, the site specifying unit, or the second distance calculation unit may be included in the ultrasound diagnostic apparatus 20 or included in the server 5. In other words, at least one of acquiring the first position, the second position, and the angle of the ultrasound probe 1, specifying the site in the ultrasound image, or calculating the first linear distance L1 and the second linear distance L2 may be performed in the ultrasound diagnostic apparatus 20 or in the server 5.

The present invention can be similarly applied to a portable ultrasound system in which an apparatus main body is realized by a laptop terminal device, and a handheld ultrasound system in which an apparatus main body is realized by a handheld terminal device such as a smartphone or a tablet personal computer (PC).

In the device of the present invention, the hardware configurations of the processing units executing various kinds of processing such as the transmission and reception circuit 14, the image generation unit 31, the display control unit 33, the distance calculation units 35 and 72, the apparatus control unit 36, and the server control unit 75 may be dedicated hardware, or may be various processors or computers that execute programs. The hardware configurations of the image memory 32 and the like may be dedicated hardware, or may be a memory such as a semiconductor memory and a storage device such as a hard disk drive (HDD) and a solid state drive (SSD).

The various processors include a central processing unit (CPU) as a general-purpose processor executing software (program) and functioning as various processing units, a programmable logic device (PLD) as a processor of which the circuit configuration can be changed after manufacturing such as a field programmable gate array (FPGA), and a dedicated electric circuit as a processor having a circuit configuration designed exclusively for executing a specific process such as an application specific integrated circuit (ASIC).

One processing unit may be configured by one of the various processors or may be configured by a combination of the same or different kinds of two or more processors, for example, a combination of a plurality of FPGAs or a combination of an FPGA and a CPU). Further, a plurality of processing units may be configured by one of various processors, or two or more of a plurality of processing units may be collectively configured by using one processor.

For example, there is a form where one processor is configured by a combination of one or more CPUs and software as typified by a computer, such as a server and a client, and this processor functions as a plurality of processing units. Further, there is a form where a processor fulfilling the functions of the entire system including a plurality of processing units by one integrated circuit (IC) chip as typified by a system on chip (SoC) or the like is used.

Furthermore, the hardware configurations of these various processors are more specifically electric circuitry where circuit elements, such as semiconductor elements, are combined.

The method of the present invention can be carried out, for example, by a program for causing a computer to execute each step of the method. Further, a computer-readable recording medium in which this program is recorded can also be provided.

The present invention has been described in detail, but the present invention is not limited to the above-described embodiments, and various improvements and changes may be made within a range not departing from the scope of the present invention.

### EXPLANATION OF REFERENCES

1: ultrasound probe

3: apparatus main body

5: server

10: ultrasound system

11: transducer array

14: transmission and reception circuit

16: signal processing unit

17: image processing unit

18: DSC

20: ultrasound diagnostic apparatus

23: magnetic sensor

28: magnetic field generator

29: magnetic field position detector

30: position detection device

32: image memory

33: display control unit

34: monitor

35: distance calculation unit

36: device control unit

37: input device

39: processor

51: pulser

52: amplification unit

53: AD conversion unit

54: beam former

60: position acquisition unit

64: first distance calculation unit

62: site specifying unit

66: second distance calculation unit

68: third distance calculation unit

72: distance calculation unit

75: server control unit

What is claimed is:

1. An ultrasound system comprising:
an ultrasound probe;
a position sensor that outputs a position detection signal for detecting a position of the ultrasound probe in a three-dimensional space;
an image generation unit that generates an ultrasound image including epidermis and a region of interest of a breast of a subject, from a reception signal obtained by performing transmission and reception of an ultrasound beam with respect to the region of interest using the ultrasound probe, on the epidermis above the region of interest;
a position acquisition unit that acquires a first position of the ultrasound probe on a nipple of the subject and a second position of the ultrasound probe on the epidermis above the region of interest in the three-dimensional space, which are detected on the basis of the position detection signal;
a site specifying unit that specifies the epidermis and the region of interest in the ultrasound image;
a first distance calculation unit that calculates a first linear distance L1 from the first position to the second position in the three-dimensional space;
a second distance calculation unit that calculates a second linear distance L2 from the region of interest to the epidermis above the region of interest in the ultrasound image; and
a third distance calculation unit that calculates a third linear distance L3 from the nipple to the region of interest in the ultrasound image on the basis of the first linear distance L1 and the second linear distance L2.

2. The ultrasound system according to claim 1,
wherein in a case where, by a first straight line from the first position to the second position, a second straight line from the region of interest to the epidermis above the region of interest in the ultrasound image, and a third straight line from the nipple to the region of interest in the ultrasound image in the three-dimensional space, a right triangle in which an angle formed by the first straight line and the second straight line is substantially a right angle is formed, the third distance calculation unit uses the Pythagoras' theorem to calculate the third linear distance L3 on the basis of the first linear distance L1 and the second linear distance L2.

3. The ultrasound system according to claim 1,
wherein in a case where, by a first straight line from the first position to the second position, a second straight line from the region of interest to the epidermis above the region of interest in the ultrasound image, and a third straight line from the nipple to the region of interest in the ultrasound image in the three-dimensional space, a right triangle in which an angle formed by the second straight line and the third straight line is substantially a right angle is formed, the third distance calculation unit uses the Pythagoras' theorem to calculate the third linear distance L3 on the basis of the first linear distance L1 and the second linear distance L2.

4. The ultrasound system according to claim 1,
wherein the position sensor outputs an angle detection signal for detecting an angle of the ultrasound probe on the epidermis above the region of interest with respect to a vertical direction in the three-dimensional space,
the position acquisition unit acquires a first angle θ1 of the ultrasound probe on the nipple with respect to the vertical direction and a second angle θ2 of the ultrasound probe on the epidermis above the region of interest with respect to the vertical direction in the three-dimensional space, which are detected on the basis of the angle detection signal, and
in a case where, by a first straight line from the first position to the second position, a fourth straight line extending from the nipple toward an inside of the subject at the first angle θ1, and a fifth straight line extending from the epidermis above the region of interest toward the inside of the subject at the second angle θ2, an isosceles triangle in which distances of the fourth straight line and the fifth straight line are equal is formed, and by the third straight line, a sixth straight line extending perpendicularly from the nipple to the fifth straight line, and a seventh straight line from an intersection between the fifth straight line and the sixth straight line to the region of interest, a right triangle in which an angle formed by the sixth straight line and the seventh straight line is a right angle is formed, the third distance calculation unit uses the Pythagoras' theorem to calculate the third linear distance L3 on the basis of the first linear distance L1, the second linear distance L2, the first angle θ1, and the second angle θ2.

5. The ultrasound system according to claim 1,
wherein the position sensor is a magnetic sensor, a GPS sensor, or an optical sensor.

6. The ultrasound system according to claim 1, further comprising:
a monitor; and
a display control unit that displays information on the third linear distance L3 on the monitor by superimposing the information on the ultrasound image including the region of interest.

7. The ultrasound system according to claim 6,
wherein the display control unit further displays information on the second linear distance L2 on the monitor by superimposing the information on the ultrasound image including the region of interest.

8. The ultrasound system according to claim 6,
wherein the site specifying unit specifies a pectoralis major muscle or chest wall of the subject in the ultrasound image,
the ultrasound system further comprises a fourth distance calculation unit that calculates a fourth linear distance L4 from the region of interest to the pectoralis major muscle or chest wall in the ultrasound image, and
the display control unit further displays information on the fourth linear distance L4 on the monitor by superimposing the information on the ultrasound image including the region of interest.

9. The ultrasound system according to claim 1, further comprising:
an ultrasound diagnostic apparatus; and
a server,
wherein the ultrasound diagnostic apparatus includes the ultrasound probe, the position sensor, and the image generation unit, and
the server includes the third distance calculation unit.

10. The ultrasound system according to claim 1, further comprising:
an input device that receives an instruction input from a user,
wherein the site specifying unit specifies at least one of the region of interest or the epidermis in the ultrasound image on the basis of the instruction input from the user.

11. The ultrasound system according to claim 1, further comprising:
an image analysis unit that analyzes the ultrasound image,
wherein the site specifying unit specifies at least one of the region of interest or the epidermis in the ultrasound image on the basis of an analysis result of the ultrasound image.

12. The ultrasound system according to claim 1,
wherein the site specifying unit has a determination model that has learned, using learning ultrasound images including a region of interest of a breast of a subject as teacher data, a relationship between the learning ultrasound image and the region of interest and epidermis included in the learning ultrasound image, and
the determination model uses the ultrasound image as an input, and specifies at least one of the region of interest or the epidermis in the ultrasound image.

13. The ultrasound system according to claim 1,
wherein information on the third linear distance L3 is transmitted to a picture archiving and communication system, and is displayed on a display device of the picture archiving and communication system.

14. A control method of an ultrasound system, the control method comprising:
outputting a position detection signal for detecting a position of an ultrasound probe in a three-dimensional space;
generating an ultrasound image including a region of interest and epidermis, from a reception signal obtained by performing transmission and reception of an ultrasound beam with respect to the region of interest using the ultrasound probe, on the epidermis above the region of interest of a breast of a subject;
acquiring a first position of the ultrasound probe on a nipple of the subject and a second position of the ultrasound probe on the epidermis above the region of interest in the three-dimensional space, which are detected on the basis of the position detection signal;
specifying the epidermis and the region of interest in the ultrasound image;
calculating a first linear distance L1 from the first position to the second position in the three-dimensional space;
calculating a second linear distance L2 from the region of interest to the epidermis above the region of interest in the ultrasound image; and
calculating a third linear distance L3 from the nipple to the region of interest in the ultrasound image on the basis of the first linear distance L1 and the second linear distance L2.

15. The ultrasound system according to claim 2,
wherein the position sensor is a magnetic sensor, a GPS sensor, or an optical sensor.

16. The ultrasound system according to claim 2, further comprising:
a monitor; and
a display control unit that displays information on the third linear distance L3 on the monitor by superimposing the information on the ultrasound image including the region of interest.

17. The ultrasound system according to claim 16,
wherein the display control unit further displays information on the second linear distance L2 on the monitor by superimposing the information on the ultrasound image including the region of interest.

18. The ultrasound system according to claim 7,
wherein the site specifying unit specifies a pectoralis major muscle or chest wall of the subject in the ultrasound image,
the ultrasound system further comprises a fourth distance calculation unit that calculates a fourth linear distance L4 from the region of interest to the pectoralis major muscle or chest wall in the ultrasound image, and
the display control unit further displays information on the fourth linear distance L4 on the monitor by superimposing the information on the ultrasound image including the region of interest.

19. The ultrasound system according to claim 2, further comprising:
an ultrasound diagnostic apparatus; and
a server,
wherein the ultrasound diagnostic apparatus includes the ultrasound probe, the position sensor, and the image generation unit, and
the server includes the third distance calculation unit.

20. The ultrasound system according to claim 2, further comprising:
an input device that receives an instruction input from a user,
wherein the site specifying unit specifies at least one of the region of interest or the epidermis in the ultrasound image on the basis of the instruction input from the user.
